(19) United States
(12) Patent Application Publication
(10) Pub. No.: US 2026/0272452 A1
(43) Pub. Date: Sep. 17, 2026

(54) APPARATUS AND METHODS FOR FORMING BARBS ON SUTURES UTILIZING MULTIPLE CUTTING BLADES

(71) Applicant: Covidien LP, Mansfield, MA (US)

(72) Inventors: Andrew E. Srenaski, Bethany, CT (US); Nicholas P. Maiorino, Branford, CT (US); David F. ONeill, Orange, CT (US)

(21) Appl. No.: 19/162,798

(22) PCT Filed: Feb. 28, 2024
(86) PCT No.: PCT/IB2024/051923

§ 371 Date: Sep. 5, 2025

Related U.S. Application Data
(60) Provisional application No. 63/450,226, filed on Mar. 6, 2023.

(51) Int. Cl. A61B 17/06; A61B 17/00

(52) U.S. Cl. CPC A61B 17/06166; A61B 2017/0042; A61B 2017/06176

(57) ABSTRACT

A barb cutting apparatus includes a suture handling assembly supporting a suture, and a knife assembly including first and second blades secured to and extending outwardly from a collet. The knife assembly is configured to simultaneously cut a first barb in the suture with the first blade and a second barb in the suture with the second blade. A method of forming barbs on a suture includes contacting an elongated body of a suture with a plurality of blades of a knife assembly, which are secured to a collet of the knife assembly, to simultaneously form a first plurality of barbs in the suture. The method further includes moving the suture relative to the plurality of blades a predetermined distance and contacting the elongated body of the suture with the plurality of blades to simultaneously form a second plurality of barbs in the suture.

### CROSS-REFERENCE TO RELATED APPLICATION

This application claims the benefit of U.S. Provisional Patent Application Ser. No. 63/450,226, filed Mar. 6, 2023, the entire content of which is incorporated herein by reference.

### FIELD

This disclosure generally relates to apparatus and methods of forming barbs on medical devices. More particularly, this disclosure relates to systems and devices having multiple cutting blades for barbing sutures, and methods of using the same.

### BACKGROUND

Barbed sutures are known for use in medical procedures. The barbed sutures may be designed to optimize tissue holding for a particular indication. For example, in some circumstances, barbs are arranged to allow passage of the barbed suture in one direction through tissue, but to resist movement of the barbed suture in an opposite direction and, in some other circumstances, barbs are arranged bi-directionally to allow passage of a first portion of the barbed suture in a first direction through tissue and passage of a second portion of the barbed suture in a second direction through the tissue. The number and/or pattern of barbs on a particular suture may be influenced by a variety of factors, such as the size of the wound and/or the strength required to hold the wound closed. For example, in some circumstances, a large number of barbs and/or a random configuration of the barbs may be desired to achieve optimal wound closure and, in other circumstances, where the wound or tissue repair needed is relatively small, a reduced number of barbs may be desired. Various methods of forming barbs include, for example, mechanical cutting, laser cutting, injection molding, stamping, extrusion, and the like.

### SUMMARY

Apparatus and methods described herein for forming barbs on a suture utilize at least two cutting blades to cut at least two barbs simultaneously in an outer surface of the suture. The apparatus and methods of this disclosure achieve a desired configuration of barbs on a suture in an efficient and cost-effective manner. These apparatus and methods include one or more of the following advantages over conventional apparatus and methods: simplified barbing process (e.g., minimizing number of steps required to produce barbs); increased speed and/or production capacity (e.g., decreased cut-to-cut time); and/or simplified tooling (e.g., retrofitting existing barbing equipment).

In accordance with aspects of this disclosure, a barb cutting apparatus includes a suture handling assembly and a knife assembly. The suture handling assembly supports a suture, and the knife assembly includes a collet, a first blade secured to and extending outwardly from the collet, and a second blade secured to and extending outwardly from the collet. The knife assembly is configured to simultaneously cut a first barb in the suture with the first blade and a second barb in the suture with the second blade.

In some aspect, the suture handling assembly includes a gripper configured to releasably hold and guide the suture. In some aspects, the suture handling assembly includes an advancement assembly configured to move the suture relative to the knife assembly. In some aspects, the suture handling assembly includes a rotation assembly configured to rotate the suture.

The barb cutting apparatus may include an energy assembly operably coupled to the knife assembly. The energy assembly may be an ultrasonic energy assembly.

In some aspects, the first and second blades of the knife assembly are generally parallel and laterally spaced from each other.

The collet may include a body and a head extending from the body. The first and second blades may be secured to the head of the collet. In some aspects, the first and second blades are disposed equidistantly from a center axis that extends through a center of the head of the collet.

In some aspects, each of the first and second blades of the knife assembly includes a tang secured to the collet, a blade body extending from the tang and outwardly of the collet, and a cutting edge disposed at a terminal end of the blade body. In some aspects, each of the first and second blades includes a cutting edge, and the cutting edges are offset different differences from the collet.

The collet may include a first blade slot configured to retain a tang of the first blade therein and a second blade slot configured to retain a tang of the second blade therein.

The first and second blades of the knife assembly may be single-bevel blades. In some aspects, each of the single bevel blades includes a primary bevel and a secondary bevel.

In some aspects, the knife assembly further includes a third blade secured to and extending outwardly from the collet.

In accordance with aspects of this disclosure, a method of forming barbs on a suture includes contacting an elongated body of a suture with a plurality of blades of a knife assembly of a barb cutting apparatus to simultaneously form a first plurality of barbs in the suture. The plurality of blades is secured to a single collet in spaced relation relative to each other. The method further includes moving the suture relative to the plurality of blades a predetermined distance, and contacting the elongated body of the suture with the plurality of blades to simultaneously form a second plurality of barbs in the suture.

The method may further include rotating the suture a predetermined angle of rotation about a suture axis of the suture after contacting the elongated body of the suture to form the first plurality of barbs and before contacting the elongate body of the suture to form the second plurality of barbs.

In some aspect, the plurality of blades includes a first blade and a second blade, and contacting the elongated body of the suture to form the first plurality of barbs includes forming a first barb and a second barb. In some other aspects, the plurality of blades includes a first blade, a second blade, and a third blade, and contacting the elongate body of the suture to form the first plurality of barbs includes forming a first barb, a second barb, and a third barb.

The method may further include applying ultrasonic energy to the knife assembly.

The details of one or more aspects of this disclosure are set forth in the accompanying drawings and the description below. Other aspects, as well as features, objects, and advantages of the aspects described in this disclosure will be apparent from the description and drawings, and from the claims.

### BRIEF DESCRIPTION OF THE DRAWINGS

Various aspects of this disclosure are described hereinbelow with reference to the drawings, which are incorporated in and constitute a part of this specification, wherein:

FIG. 1 is a side view of a barbed suture formed in accordance with aspects of this disclosure;

FIG. 2 is a schematic view of a barb cutting apparatus in accordance with aspects of this disclosure;

FIG. 3 is a close-up view of a suture handling assembly and a cutting assembly of the barb cutting apparatus of FIG. 2, the suture handling assembly shown gripping a suture;

FIG. 4 is a perspective view, with parts separated, of a knife assembly of the cutting assembly of FIG. 3;

FIGS. 5 and 6 are perspective views of the knife assembly of FIG. 4;

FIG. 7 is a side view of the knife assembly of FIGS. 4-6;

FIG. 8 is an end view of the knife assembly of FIGS. 4-7, shown positioned adjacent to a suture;

FIG. 9 is a front view of a first blade and a second blade of the knife assembly of FIG. 4;

FIG. 10 is a side view of the first blade and the second blade of FIG. 9;

FIG. 11 is a close-up view of the areas of detail 1 la and 11b indicated in FIG. 10;

FIG. 12 is a perspective view of a suture including barbs formed with the barb cutting apparatus of FIG. 2 in accordance with aspects of this disclosure;

FIG. 13 is a perspective view of a knife assembly suitable for use with the barb cutting apparatus of FIG. 2 in accordance with aspects of this disclosure; and

FIG. 14 is an end view of the knife assembly of FIG. 13.

### DETAILED DESCRIPTION

Aspects of this disclosure will now be described in detail with reference to the drawing figures wherein like reference numerals identify similar or identical elements. Throughout this description, the term “proximal” refers to a portion or component of a structure that is closer to a user, and the term “distal” refers to a portion or component of the structure that is farther from the user. The terms “about,” “generally,” “substantially,” and the like, shall be understood as words of approximation that take into account relatively little to no variation in the modified terms (e.g., generally accepted manufacturing, use, environmental, or measurement tolerances differing, for example, by less than 10%).

It should be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

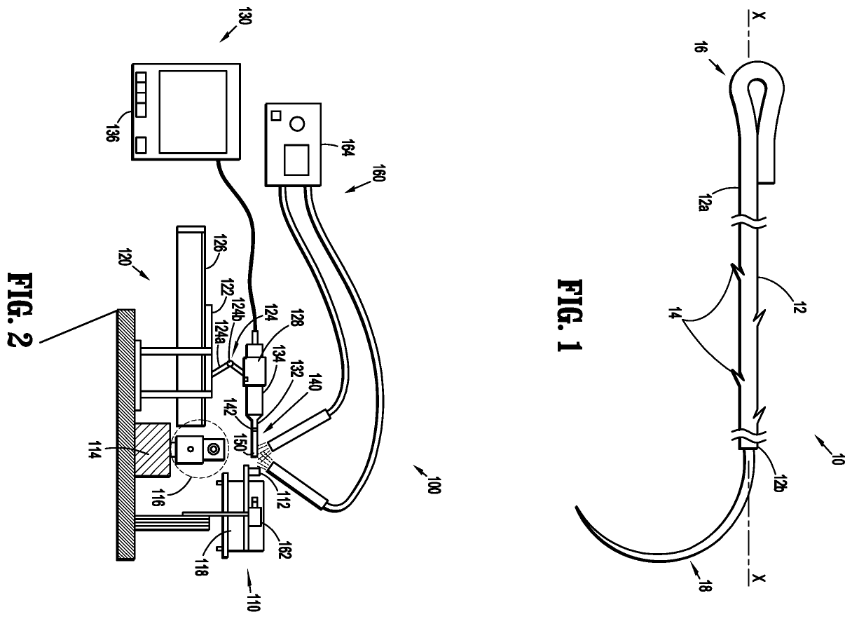

FIG. 1 illustrates a barbed suture 10 formed in accordance with a method of this disclosure. The barbed suture 10 includes an elongate body 12 extending along a suture axis “X” and barbs 14 extending outwardly from the elongate body 12. The elongate body 12 may be formed from absorbable material(s), non-absorbable material(s), and combinations thereof. The elongate body 12 may be formed using any technique within the purview of those skilled in the art, such as, for example, extrusion, molding, and/or solvent casting.

While the elongate body 12 is shown as a monofilament suture body, it should be understood that the elongate body 12 may be a multifilament suture body. The elongate body 12 may be a yarn made of multiple filaments of the same or different materials. A multifilament suture body may be formed using any known technique such as, for example, braiding, weaving, or knitting. The filaments may also be combined to produce a non-woven suture. The filaments themselves may be drawn, oriented, crinkled, twisted, commingled, or air entangled to form yarns as part of the suture forming process. Further, while the elongate body 12 is shown having a substantially circular cross-sectional shape, it should be understood that the elongate body 12 may have any suitable or desired shape.

The barbed suture 10 may include a loop 16 at a first or distal end 12a of the elongate body 12. The loop 16 may be formed and/or attached in any manner within the purview of those skilled in the art prior to or upon completion of the barb forming process and be of any size and configuration. In some aspects, the loop 16 is configured to enhance retention of the barbed suture 10 in body tissue. The barbed suture 10 may include a needle 18 attached to a second or proximal end 12b of the elongate body 12. The needle 18 may be attached to the elongate body 12 prior to or upon completion of the barb forming process.

Optionally, the barbed suture 10 may be coated or impregnated with one or more bioactive agents (e.g., to accelerate or beneficially modify the healing process when the barbed suture is applied to a wound or surgical site) and/or additives, such as dyes, pigments, or colorants (e.g., to increase visibility of the barbed suture in the surgical field). The bioactive agents and/or the additives may be applied onto the barbed suture 10 utilizing any method within the purview of those skilled in the art including, for example, dipping, spraying, vapor deposition, brushing, mixing, compounding, and the like.

The barbs 14 extend along at least a portion of the length of the elongate body 12 in spaced relation relative to each other. As shown, the barbs 14 are disposed in a helical pattern around the elongate body 12. It should be understood, however, that other patterns are envisioned and the pattern of the barbs 14 may be consistent along the length of the elongate body 12 (e.g., be symmetrical) or may vary (e.g., be asymmetrical or random). Further, the barbs 14 are shown formed projecting from the elongate body 12 toward the first end 12a of the barbed suture 10. It should be understood that the barbs 14 may be formed such that some of the barbs 14 project toward the first end 12a and the remaining barbs 14 project toward the second end 12b so as to form a bi-directional suture. Further still, while the barbs 14 are shown as all being the same, it should be understood that the barbs may be different (single and/or compound, small and/or large, etc.). Accordingly, the barbs 14 may be formed in any number, size, configuration, spacing, and/or orientation depending upon the desired characteristics of the barbed suture 10 for its intended use (e.g., the type of tissue being manipulated and/or the procedure performed), as well as the composition and/or geometry of the material utilized to form the barbed suture 10.

FIG. 2 illustrates a barb cutting apparatus 100 in accordance with aspects of this disclosure for forming the barbs 14 (FIG. 1) on a barbed suture 10 (also referred to herein generally as a suture in reference to the barbed suture prior to the barbs being formed thereon). The barb cutting apparatus 100 includes a suture handling assembly 110 and a cutting assembly 120. In some aspects, the barb cutting apparatus 100 includes a visual inspection assembly 160.

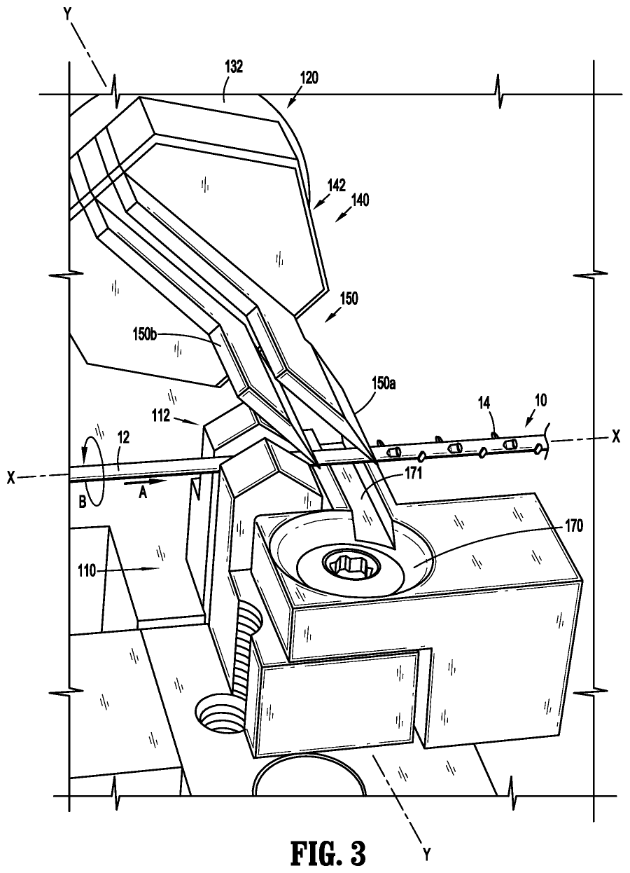

With reference to FIGS. 2 and 3, the suture handling assembly 110 is configured to hold and guide one or more sutures 10 in the barb cutting apparatus 100. The suture handling assembly 110 includes a gripper 112 for supporting the suture 10 in a fixed position. The suture handling assembly 110 includes an advancement or indexing assembly 114 configured to move the suture 10 linearly through the gripper 112 (e.g., in the direction of arrow “A” seen in FIG. 3) along the suture axis “X” that is generally perpendicular to a central axis “Y” (FIG. 3) of the cutting assembly 120. The advancement assembly 114 moves the suture 10 a predetermined distance or increment after each cut is made to form barbs 14 in the suture 10 for appropriate axial barb spacing or barb pitch. The suture handling assembly 110 includes a rotation assembly 116 configured to rotate the suture 10 about the suture axis “X” (e.g., in the direction of arrow “B” seen in FIG. 3) after each cut is made for appropriate radial barb spacing. In some aspects, the suture handling assembly 110 further includes a motorized slide 118 configured to move the suture 10 three-dimensionally (e.g., in X, Y, and Z planes) relative to the cutting assembly 120.

The cutting assembly 120 is configured for cutting barbs 14 in the elongate body 12 of a suture 10. The cutting assembly 120 includes an energy assembly 130 and a knife assembly 140 operably coupled to a base 122. In aspects, the cutting assembly 120 includes a knife positioning arm 124 secured to and extending from the base 122. The knife positioning arm 124 supports the energy and knife assemblies 130, 140. In some aspects, the knife positioning arm 124 includes one or more arm links 124a interconnected at arm joints 124b. In some aspects, the knife positioning arm 124 may utilize linkages, cams, followers, rollers, or the like that are configured to move the knife assembly 140 three-dimensionally and/or along a barb cutting path. In aspects, the cutting assembly 120 includes a knife positioning slide 126 for adjusting the position of the knife assembly 140 relative to the suture handling assembly 110. In some aspects, the cutting assembly 120 further includes a rotary mechanism 128 for rotating the knife assembly 140, for example, to adjust the cut angle of the barbs 14.

In aspects, the energy assembly 130 is an ultrasonic energy assembly including a horn 132, a converter 134, and an ultrasonic generator 136. The horn 132 is configured and dimensioned to accept the knife assembly 140 for forming barbs 14 on the suture 10. The converter 134 is operably coupled to the horn 132 and transmits ultrasonic energy to the horn 132. The converter 134 converts electrical energy to mechanical energy which causes displacement of the knife assembly 140 at a predetermined ultrasonic frequency powered by the ultrasonic generator 136. The ultrasonic generator 136 may be manipulated to either increase or decrease the ultrasonic frequency which may be transmitted to the knife assembly 140. Additionally or alternatively, the energy assembly 130 may include a heating element (not shown) or other suitable mechanism for heating the knife assembly 140. By vibrating and/or heating the knife assembly 140, the force necessary to cut the barbs 14 into the suture 10 may be reduced.

The knife assembly 140 includes a base or collet 142 and blades 150 secured to, and extending outwardly from, the collet 142. The collet 142 is configured to releasably engage (e.g., be received within) the horn 132 and fixedly connect the blades 150 to the energy assembly 130. While the knife assembly 140 is shown and described as including two blades 150, a first blade 150a and a second blade 150b, it should be understood that the knife assembly 140 may include more than two blades 150.

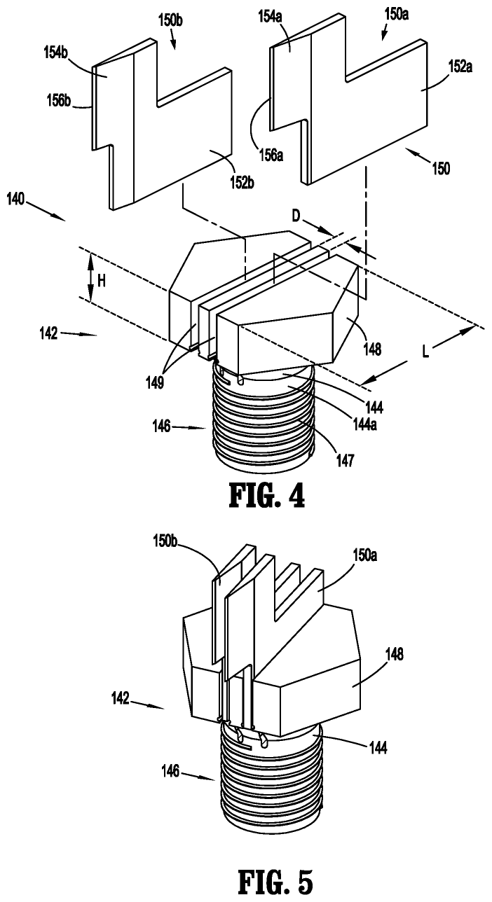

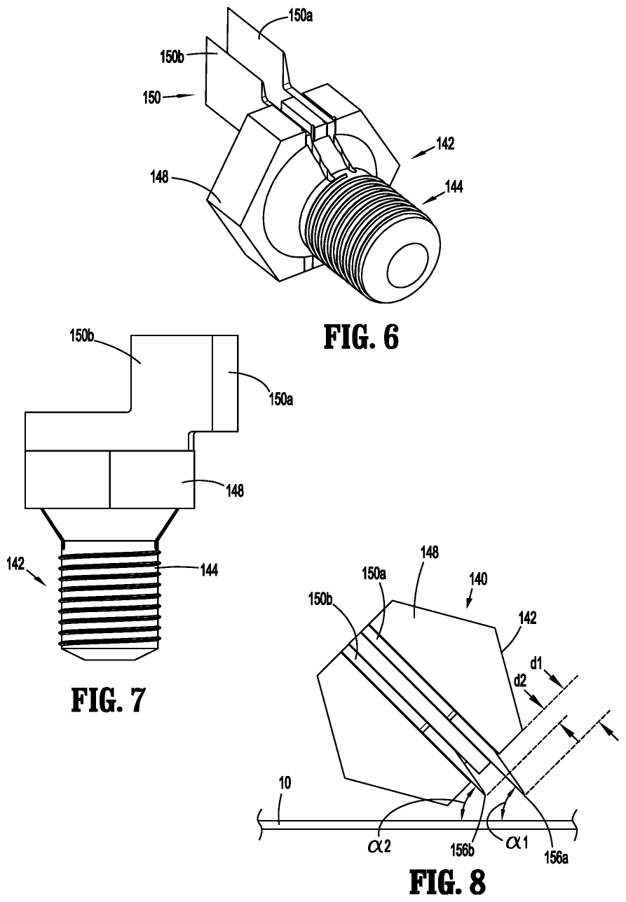

As shown in FIGS. 4-8, the collet 142 includes a body 144 including a connection region 146 configured to releasably engage the horn 132 (FIG. 3) and a head 148 extending from the body 144 configured to fixedly engage the blades 150. In aspects, the connection region 146 of the collet 142 includes threads 147 disposed on an external surface 144a of the body 144 that are configured to mate with threads (not shown) defined in a recess of the horn 132. It should be understood that other mating configurations between the collet 142 and the horn 132 (e.g., other threaded couplers, bayonet couplers, push-pull connectors, etc.) may be utilized for detachably coupling the knife assembly 140 to the energy assembly 130.

Blade slots 149 are defined in the head 148, and each of the blade slots 149 is sized and shaped to receive and retain a tang 152a, 152b of the first or second blade 150a, 150b in a secure fixed manner. The blade slots 149 are disposed equidistantly from the center axis “Y” (FIG. 3) of the cutting assembly 120 that extends through the center of the head 148. The blade slots 149 are laterally spaced relative to each other a predetermined distance “D” corresponding to the desired spacing of the blades 150 and thus, the desired axial barb spacing. In some aspects, to maximize the surface contact between the collet 142 and the blades 150, the blade slots 149 extend a majority of a length “L” and/or a majority of a height “H” of the head 148 of the collet 142, and in certain aspects, the entire length “L” and/or the entire height “H” of the head 148. It should be understood that the shape of the head 148 and/or the spacing of the blade slots 149 may vary depending upon, for example, the desired number of blades, position of the blades, spacing between the blades, etc. Further, it should be understood that other connection mechanisms between the head 148 and the blades 150 are envisioned.

Each of the first and second blades 150a, 150b includes a tang 152a, 152b configured to be fixedly received with a respective blade slot 149 of the collet 142 and a blade body 154a, 154b extending from the tang 152a, 152b and having a cutting edge 156a, 156b defined at a terminal end of the blade body 154a, 154b for cutting barbs in sutures. As specifically seen in FIG. 8, the cutting edges 156a, 156b of the first and second blades 150a, 150b are offset different distances “d1, “d2” from the head 148 of the collet 142 to facilitate the correct positioning of the cutting edges 156a, 156b relative to the suture 10 when the first and second blades 150a, 150b are disposed at non-zero cutting angles with respect to the suture 10 (e.g., both the first and second blades 150a, 150b are aligned to cut the suture 10 at the same time). This offset may also ensure proper backlighting of the first and second blades 150a, 150b for proper imaging of formed barbs by the visual inspection assembly 160 (FIG. 2).

With continued reference to FIG. 8, the cutting angle “α1,” “α2” of the first and second blades 150a, 150b relative to the suture 10 may be fixed. In aspects, the cutting angles “α1,” “α2” are non-zero angles and, in some aspects, the cutting angles “α1,” “αv2” are acute angles and, in certain aspects, the cutting angles “α1,” “α2” are about 45° relative to the suture 10. In some aspects, the cutting angles “α1,” “α2” may be adjusted during the barb forming process by rotating the knife assembly 140 (e.g., via the rotary mechanism 128 (FIG. 2)) relative to the suture 10 to form, for example, a compound cut or barbs of different shapes and/or sizes along the suture 10.

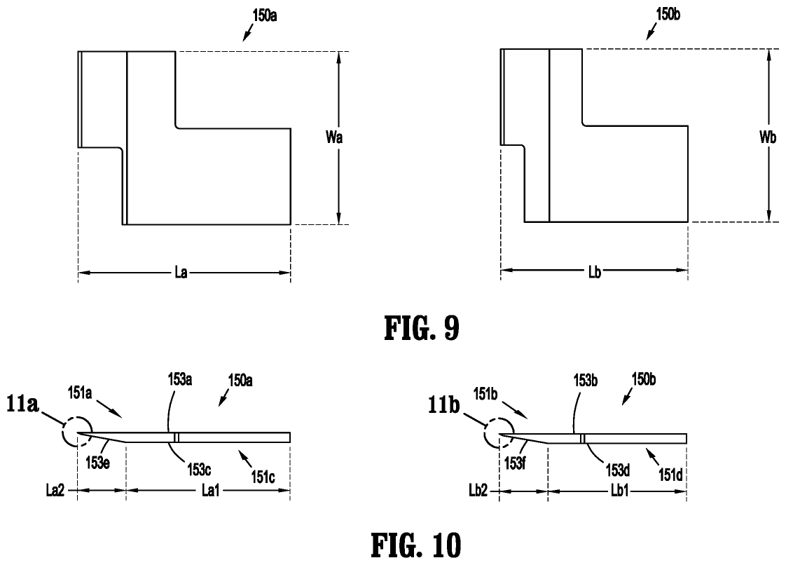

FIGS. 9-11 illustrate aspects of fabricating the first and second blades 150a, 150b to maximize the clamping area of the first and second blades 150a, 150b in the collet 142 (FIG. 4) and to minimize the weight of the knife assembly 140. As seen in FIG. 9, each of the first and second blades 150a, 150b define an overall length “La,” “Lb” and an overall width “Wa,” “Wb.” In aspects, the overall width “Wa,” “Wb” of the first and second blades 150a, 150b are substantially the same. In aspects, the overall length “La” of the first blade 150a, which is positioned farther from the suture 10 (FIG. 8) is greater than the overall length “Lb” of the second blade 150b, which is positioned closer to the suture 10. Such a configuration enables the first and second blades 150a, 150b to be fabricated with different offsets, as discussed above.

As seen in FIG. 10, the first and second blades 150a, 150b are single-bevel blades. Each of the first and second blades 150a, 150b includes a first or suture facing side 151a, 151b having a generally planar surface 153a, 153b along the overall length “La,” “Lb” (FIG. 9) of the first and second blades 150a, 150b. A second side 151c, 151d, opposite the first side 151a, 151b and that faces away from the suture 10 (FIG. 8), has a generally planar first portion 153c, 153d along a length “La1,” “Lb1” of the tang 152a, 152b, and a tapered second portion 153e, 153f along a length “La2,” “Lb2” of the blade body 154a, 154b.

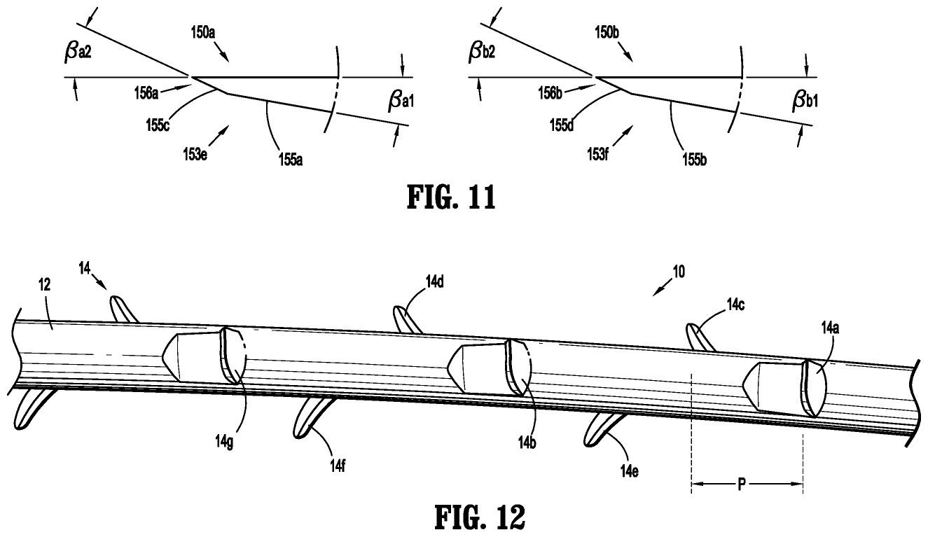

As seen in FIG. 11, the tapered second portion 153e, 153f of the first and second blades 150a, 150b includes a primary bevel 155a, 155b and a secondary bevel 155c, 155d. The primary bevel 155a, 155b extends a majority of the length “La2,” “Lb2” (FIG. 10) of the blade body 154a, 154b and in aspects, has a primary bevel angle “ßα1,” “ßb1” of about 2° to about 20° and, in some aspects, a primary bevel angle “ßa1,” “ßb1” of about 5° to about 15°, and in certain aspects, a primary bevel angle “ßa1,” “ßb1” of about 10°. The secondary bevel 155c, 155d defines the cutting edge 156a, 156b and in aspects, has a secondary bevel angle “ßa2,” “ßb2” of about 15° to about 35° and, in some aspects, a secondary bevel angle “ßa2,” “ßb2” of about 20° to about 30°, and in certain aspects, a secondary bevel angle “ßa2,” “ßb2” of about 25°. It should be understood, however, that the bevel angles of the first and second blades may be adjusted depending on the desired barb configuration.

With reference again to FIG. 2, the visual inspection assembly 160 includes at least one camera 162 configured for monitoring the cutting of the barbs 14 (FIG. 1) in the suture 10 and a lighting system 164 configured for optimizing the view of the camera 162. In aspects, the camera 162 records the method of forming barbs. In some aspects, in the event that a mis-formed or defective barb is cut, the visual inspection assembly 160 may be configured to provide an alert to signal that a defect has occurred. In certain aspects, the visual inspection assembly 160 is configured to signal a suture severing assembly (not shown) to sever the defective suture.

With continued reference to FIG. 2, in a method of operation, a suture 10 (FIG. 3) is secured to the suture handling assembly 110 and the visual inspection assembly 160 is activated. The suture handling assembly 110 and the cutting assembly 120 are positioned relative to each other such that the blades 150 of the knife assembly 140 are positioned in close proximity to (but not in contact with) the suture 10 (e.g., via the knife positioning slide 126). The energy assembly 130 is then activated to cause the ultrasonic vibrating or heating of the blades 150. Once the energy assembly 130 has been activated, barbs 14 are formed on the suture 10 by moving the cutting assembly 120 relative to the suture 10. In aspects, the blades 150 of the cutting assembly 120 are brought into contact with the suture 10 (e.g., via the knife positioning arm 122) while the suture 10 remains in a stationary or fixed position during the barb forming or cutting process. In some aspects, the suture 10 is moved (e.g., via the advancement assembly 114 and/or the rotation assembly 116) between each of the barb cuts. In some other aspects, the blades 150 are moved (e.g., via the knife positioning arm 122 and/or the rotary mechanism 128) between each of the barb cuts.

Alternatively, the barbs 14 may be formed on the suture 10 by movement of the suture handling assembly 110 relative to the cutting assembly 120. In this manner, the suture 10 is moved into contact with the blades 150 of the knife assembly 140 (e.g., via the motorized slide 118) while the knife assembly 140 remains in a stationary or fixed position during the barb forming process.

Initially, a first plurality of the barbs 14 are simultaneously (or substantially simultaneously) formed in the suture 10 by moving the blades 150 along a predetermine barb cutting path and using ultrasonic energy at various frequencies and signal amplitudes to cut the elongate body 12 of the suture 10. The suture 10 is then moved (e.g., rotated and/or advanced) relative to the blades 150 before a second plurality of the barbs 14 are formed in the suture 10 by moving the blades 150 again to cut the elongate body 12 of the suture 10. This process is repeated to form the desired configuration of barbs 14 on the suture 10.

With reference again to FIG. 3, in conjunction with FIG. 12, the barb cutting apparatus 100 (FIG. 2) is set-up to form a barbed suture 10 having a barb configuration which includes the barbs 14 occurring every 120° along the elongate body 12 of the suture 10 at a consistent barb pitch “P” along the length of the suture 10 utilizing a knife assembly 140 including two blades 150. To accommodate the orientation and pitch requirements, the cutting of two barbs 14 simultaneously with two blades 150 requires the blades 150 to be spaced apart by about three times the barb pitch “P” (e.g., which is set by the distance “D” (FIG. 4) of the blades slots 149 from one another) to cut the barbs 14 on the same 120° plane.

A first barb cutting sequence is initiated by moving the blades 150 towards the suture 10 and cutting a first barb 14a into the suture 10 by the first blade 150a and a second barb 14b into the suture 10 by the second blade 150b. The first and second barbs 14a, 14b are axially spaced and adjacent to one another at three times the barb pitch “P.” After the first and second barbs 14a, 14b are formed, the suture 10 is then advanced by one times the barb pitch “P” and rotated 120°about the suture axis “X” (advancement and rotation may occur in any order or simultaneously). As seen in FIG. 3, a cutting floor 170 of the barb cutting apparatus 100 (FIG. 2) includes a channel 171 disposed below the first and second blades 150a, 150b that is sized (e.g., two times the barb pitch “P”) to accommodate the advancement and rotation of the suture 10 to allow the formed barbs 14 to rotate past the cutting floor 170 without interference. After the desired movement of the suture 10, the blades 150 are moved again towards the suture 10 to cut a third barb 14c into the suture 10 by the first blade 150a and a fourth barb 14d into the suture 10 by the second blade 150b. After the third and fourth barbs 14c, 14d are formed, the suture 10 is then advanced by one times the barb pitch “P” and rotated 120°about the suture axis “X.” After the suture 10 is moved relative to the blades 150, the blades 150 are moved against towards the suture 10 to cut a fifth barb 14e into the suture 10 by the first blade 150a and a sixth barb 14f into the suture 10 by the second blade 150b. After the fifth and sixth barbs 14e, 14f are formed, the suture 10 is then advanced by four times the barb pitch “P” and rotated 120° about the suture axis “X” to repeat this barb cutting sequence a second time to cut the next six barbs.

Additional barbs 14 are formed in the manner described above (e.g., by performing the barb cutting sequence an appropriate number of times). The process continues until the barbs 14 are formed along the desired length of the elongate body 12 of the suture 10. The barbed suture 10 is then unloaded from the barb cutting apparatus 10 as the barb forming process is complete.

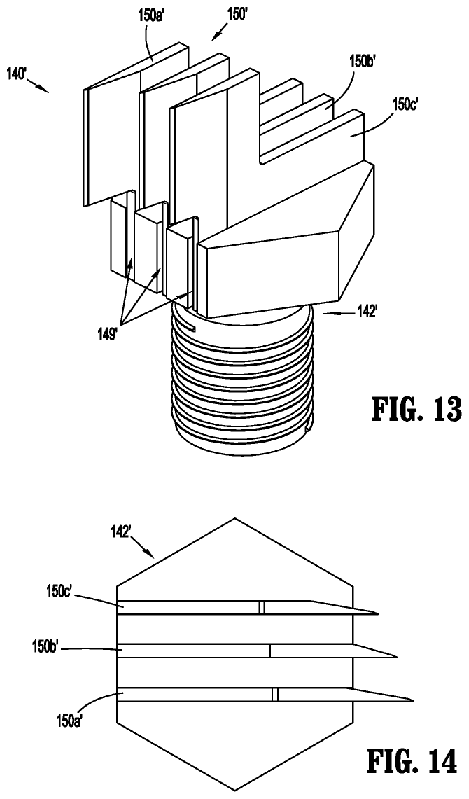

While the knife assembly has been described as including a first blade and a second blade, the knife assembly may have more than two blades (e.g., three blades, four blades, five blades, etc.) secured to a collet that is configured to support the desired number and configuration of the blades. For example, as shown in FIGS. 13 and 14, a knife assembly 140′ may include a first blade 150a′, a second blade 150b′, and a third blade 150c′ supported within a collet 142′. The knife assembly 140′ is substantially the same as the knife assembly 140 of FIG. 5, with modifications for the inclusion of the third blade 150c′ (for example, the arrangement and inclusion of an additional blade slot 149′).

Further, it should be understood that the sequence and cutting pattern of barbs in a suture is dependent upon the number of blades used and the desired configuration and orientation of the barbs. For example, when using the knife assembly 140′ including three blades 150′, as seen in FIG. 13, to form a barbed suture 10 including the barb configuration seen in FIG. 8, the first cut into the suture 10 forms the first barb 14a, the second barb 14d, and a seventh barb 14g (as identified in FIG. 8) by the first blade 150a′, the second blade 150b', and the third blade 150c′, respectively, and the advancement pattern between each cut would be one times the barb pitch “P,” one times the barb pitch “P,” and then seven times the barb pitch “P” to repeat the barb cutting sequence, with a 120° rotation between each cut. As another example, when using a knife assembly including four blades to form the barbed suture including the barb configuration seen in FIG. 8, the first cut into the suture with the four blades forms the first barb 14a, the second barb 14b, the seventh barb 14g, and an eighth barb (not shown) and the advancement pattern between each cut would be one times the barb pitch “P,” one times the barb pitch “P,” and then ten times the barb pitch “P” to repeat the barb cutting sequence, with a 120° rotation between each cut. As yet another example, when using a knife assembly including five blades to form the barbed suture including the barb configuration seen in FIG. 8, the first cut into the suture with the five blades forms the first barb 14a, the second barb 14b, the seventh barb 14g, an eighth barb (not shown), and a ninth barb (not shown), and the advancement pattern between each cut would be one times the barb pitch “P,” one times the barb pitch “P,” and then thirteen times the barb pitch “P” to repeat the barb cutting sequence, with a 120° rotation between each cut. This method can be adapted to cut barbs in any orientation along a suture.

Further still, other configurations of the knife assembly are envisioned. For example, while the blades are shown and described as being linear blades, the blades may have other shapes (e.g., curved). As another example, the position of the blades in the collet, the spacing between the blades, the offset of the blades relative to the collet, etc. may vary depending upon the desired barb configuration. As yet another example, while the blades of the knife assembly are shown as all being substantially the same, the blades may be different to form barbs having different shapes and/or sizes (e.g., the cutting edges may be modified to change the cut depth, cut angle, etc.).

While this disclosure describes apparatus and methods of forming barbs on sutures, it should be understood that barbs may be formed on other medical devices such as, for example, surgical fibers, anchors, slit sheets, ribbons, tapes, meshes, stents, scaffolds, pledgets, and vascular grafts.

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be made by one skilled in the art without departing from the scope or spirit of the disclosure. Additionally, the elements and features shown and described in connection with certain aspects of the disclosure may be combined with the elements and features of certain other aspects without departing from the scope of the disclosure, and that such modifications and variation are also included within the scope of the disclosure. Therefore, the above description should not be construed as limiting, but merely as exemplifications of aspects of the disclosure. Thus, the scope of the disclosure should be determined by the appended claims and their legal equivalents, rather than by the examples given.

What is claimed is:

1. A barb cutting apparatus, comprising:
a suture handling assembly supporting a suture; and
a knife assembly including:
a collet;
a first blade secured to and extending outwardly from the collet; and
a second blade secured to and extending outwardly from the collet,
the knife assembly configured to simultaneously cut a first barb in the suture with the first blade and a second barb in the suture with the second blade.

2. The barb cutting apparatus according to claim 1, wherein the suture handling assembly includes a gripper configured to releasably hold and guide the suture.

3. The barb cutting apparatus according to claim 1, wherein the suture handling assembly includes an advancement assembly configured to move the suture relative to the knife assembly.

4. The barb cutting apparatus according to claim 1, wherein the suture handling assembly includes a rotation assembly configured to rotate the suture.

5. The barb cutting apparatus according to claim 1, further comprising an energy assembly operably coupled to the knife assembly.

6. The barb cutting apparatus according to claim 5, wherein the energy assembly is an ultrasonic energy assembly.

7. The barb cutting apparatus according to claim 1, wherein the first and second blades are generally parallel and laterally spaced from each other.

8. The barb cutting apparatus according to claim 1, wherein the collet includes a body and a head extending from the body, and the first and second blades are secured to the head of the collet.

9. The barb cutting apparatus according to claim 8, wherein the first and second blades are disposed equidistantly from a center axis that extends through a center of the head of the collet.

10. The barb cutting apparatus according to claim 1, wherein each of the first and second blades includes a tang secured to the collet, a blade body extending from the tang and outwardly of the collet, and a cutting edge disposed at a terminal end of the blade body.

11. The barb cutting apparatus according to claim 1, wherein each of the first and second blades includes a cutting edge, and the cutting edges are offset different differences from the collet.

12. The barb cutting apparatus according to claim 1, wherein the collet includes a first blade slot configured to retain a tang of the first blade therein and a second blade slot configured to retain a tang of the second blade therein.

13. The barb cutting apparatus according to claim 1, wherein the first and second blades are single-bevel blades.

14. The barb cutting apparatus according to claim 13, wherein each of the single bevel blades includes a primary bevel and a secondary bevel.

15. The barb cutting apparatus according to claim 1, wherein the knife assembly further includes a third blade secured to and extending outwardly from the collet.

16. A method of forming barbs on a suture, the method comprising:
contacting an elongated body of a suture with a plurality of blades of a knife assembly of a barb cutting apparatus to simultaneously form a first plurality of barbs in the suture, the plurality of blades secured to a single collet in spaced relation relative to each other;
moving the suture relative to the plurality of blades a predetermined distance; and
contacting the elongated body of the suture with the plurality of blades to simultaneously form a second plurality of barbs in the suture.

17. The method according to claim 16, further comprising rotating the suture a predetermined angle of rotation about a suture axis of the suture after contacting the elongated body of the suture to form the first plurality of barbs and before contacting the elongate body of the suture to form the second plurality of barbs.

18. The method according to claim 16, wherein the plurality of blades includes a first blade and a second blade, and contacting the elongated body of the suture to form the first plurality of barbs includes forming a first barb and a second barb.

19. The method according to claim 16, wherein the plurality of blades includes a first blade, a second blade, and a third blade, and contacting the elongate body of the suture to form the first plurality of barbs includes forming a first barb, a second barb, and a third barb.

20. The method according to claim 16, further comprising applying ultrasonic energy to the knife assembly.
